(19) United States
(12) Patent Application Publication
(10) Pub. No.: US 2015/0232943 A1
(43) Pub. Date: Aug. 20, 2015

(54) METHODS OF DIAGNOSING LIVER CANCER IN A SUBJECT AND A KIT FOR DIAGNOSING LIVER CANCER

(71) Applicant: SINGAPORE HEALTH SERVICES PTE LTD, Singapore (SG)

(72) Inventor: Kam Man Hui, Singapore (SG)

(73) Assignee: SINGAPORE HEALTH SERVICES PTE LTD, Singapore (SG)

(21) Appl. No.: 14/429,317

(22) PCT Filed: Sep. 23, 2013
(86) PCT No.: PCT/SG2013/000414

§ 371 Date: Mar. 18, 2015

Related U.S. Application Data
(60) Provisional application No. 61/704,425, filed on Sep. 21, 2012.

(51) Int. Cl. C12Q 1/68; G01N 33/574

(52) U.S. Cl. CPC C12Q 1/6886; G01N 33/57438; C12Q 2600/158; G01N 2800/50

(57) ABSTRACT

Disclosed are methods of diagnosing liver cancer in a subject as well as methods of assessing the risk of a subject having chronic hepatitis and liver cirrhosis of developing liver cancer. Also disclosed are kits for the diagnosis of liver cancer.

### CROSS-REFERENCE TO RELATED APPLICATIONS

The present invention claims the benefit of priority to U.S. Provisional Patent Application No. 61/704,425, titled “METHODS OF DIAGNOSING LIVER CANCER IN A SUBJECT AND A KIT FOR DIAGNOSING LIVER CANCER” filed on Sep. 21, 2012, the entire disclosure of which is hereby incorporated by reference in its entirety for all purposes.

### FIELD OF THE INVENTION

The present invention claims relates to a method of diagnosing liver cancer in a subject as well as to method of assessing the risk of a subject having liver cirrhosis of developing liver cancer. The invention also relates to kits for the diagnosis of liver cancer.

### BACKGROUND OF THE INVENTION

Hepatocellular carcinoma (HCC) is one of the most common cancers worldwide and the third most frequent cause of cancer death, with an annual incidence of more than 500 thousand cases worldwide (Kamangar et al (2006). J Clin Oncol, 24, 2137-50; Boyle P. (2008). Annals of Oncology, 19:605-606). The outcome of HCC patients remains poor as a result of late diagnosis. Currently, serum α-fetoprotein (AFP) level and ultrasonography are commonly used for HCC screening and diagnosis. However, the clinical usefulness of this approach is limited for several reasons. First, AFP is not elevated in all HCC patients, and maybe elevated by chronic liver disease, leading to unsatisfactory sensitivity and specificity. At a cut-off value at 20 ng/ml, the sensitivity ranges from 41% to 64% reported by different studies, and specificity from 80% to 91% (Daniele et al, (2004) Gastroenterology. 2004 November; 127 (5 Suppl 1):S108-12. According to the American Association for the Study of Liver Diseases (AASLD) practice guidelines published in 2005, 200 ng/ml was recommended to be the diagnostic cut-off point, with a sensitivity of 22% and more than 99% specificity (Trevisani et al, J Hepatol. (2001) April; 34(4):570-5, Lok et al., Gastroenterology (2010) February; 138(2):493-502). In 2010, based on the results from recent studies, the 2010 AASLD guideline for HCC management recommend ultrasound alone for surveillance, and no longer includes AFP for both surveillance and diagnosis Bruix & Sherman, Hepatology (2011) March; 53(3):1020-22). On the other hand, ultrasound has its own limitation. It is difficult to detect tumours in the cirrhotic liver that have massive abnormality. In addition, its performance highly depends on the operators experience and sophistication of the equipment, and it may not be available to those who live in underdeveloped areas. For these reasons, much efforts have been put into searching for more reliable markers for HCC screening and diagnosis.

The ideal maker for HCC should be both specific and sensitive, and is from specimens that have easy accesses. With the development of high density microarray and proteomics, many new markers have been identified in recent years. One initial exploratory approach is to look for leads in HCC tumour tissues, such as glypican 3 (GPC3) and Golgi protein 73 (GP73) (Liu et al., World J Gastroenterol. (2010) Sep. 21; 16(35):4410-5, Capurro et al, Gastroenterology. (2003) July; 125(1):89-97) and validate its presence in peripheral blood by ELISA or Western blot. Some studies use mass spectrometry to profile proteins in plasma to identify protein marker, such as osteopontin (OPN) (Shang et al, Hepatology. (2012) February; 55(2):483-90). Other studies utilize microarray to profile nucleic acid from plasma or serum to identify gene marker or microRNA markers (Zhou et al, J Clin Oncol. 2011 Dec. 20; 29(36):4781-8.). Among the novel markers for HCC, the most extensively studied markers are des-gamma-carboxyprothrombin (DCP) and glyco form of AFP (AFP-L3). Although the sensitivity of DCP was reported to be relatively better (74%), but the specificity is unsatisfactory (70-86%) (Marerro et al, Gastroenterology. (2009) July; 137(1):110-8, Lok et al, supra). It was concluded that neither AFP nor DCP is optimal to complement ultrasound in detection of early HCC (Lok et al, supra).

Thus, there is need to find new markers that are suitable for the early detection of HCC.

### SUMMARY OF INVENTION

The present invention provides a method of diagnosing liver cancer in a subject. The method comprises determining in a sample obtained from the subject the gene expression level of at least one marker gene selected from the group consisting of the tumor necrosis factor, alpha-induced protein 3 (TNFAIP3) gene, the amphiregulin (AREG) gene and the GTPase, IMAP family member 5 (GIMAP5) gene.

The present invention also provides a method of assessing the risk of a subject having liver cirrhosis of developing liver cancer. This method comprises determining in a sample obtained from the subject the gene expression level of at least one marker gene selected from the group consisting of the tumor necrosis factor, alpha-induced protein 3 (TNFAIP3) gene, the amphiregulin (AREG) gene and the GTPase, IMAP family member 5 (GIMAP5) gene.

The present invention further provides a method of diagnosing liver cancer in a subject. This method comprises determining in the sample obtained from the subject the presence or amount of at least one marker protein selected from the group consisting of tumor necrosis factor, alpha-induced protein 3 (TNFAIP3, SwissProt accession number: P21580), amphiregulin (AREG, SwissProt accession number P15514) and GTPase, IMAP family member 5 (GIMAP5, SwissProt accession number Q96F15).

The present invention also provides a kit for the diagnosis of liver cancer by determining the expression level of at least one marker gene selected from the group consisting of the tumor necrosis factor, alpha-induced protein 3 (TNFAIP3) gene, the amphiregulin (AREG) gene and the GTPase, IMAP family member 5 (GIMAP5) gene. The kit comprises one or more oligonucleotides complementary to at least one of the marker gene nucleic acid molecule.

### BRIEF DESCRIPTION OF THE DRAWINGS

The invention will be better understood with reference to the detailed description when considered in conjunction with the non-limiting examples and the accompanying drawings, in which:

FIG. 1 shows the study design used in the present invention. A group of 28 individuals recruited at Sun Yat-sen University Cancer Center, GuangZhou 8^{th} People's Hospital (China), were used in the initial discovered set. These 28 patients comprised 10 patients diagnosed with HCC, 12 patients diagnosed with chronic hepatitis and 6 healthy patients. A high density gene microarray was used to profile gene expression in the white blood cells (WBC) isolated from HCC patients and chronic Hepatitis patients, and healthy individuals. After the initial gene screening, a group of 50 patients diagnosed with HCC, 50 patients diagnosed with chronic hepatitis also recruited at Sun Yat-sen University Cancer Center, GuangZhou 8^{th} People's Hospital, were used to establish a training set and to develop a 3-gene logistic model. This model was validated in an independent cohort of 60 patients diagnosed with both HBV and HCC and 90 patients with chronic Hepatitis (CHB patients) recruited at the Singapore General Hospital and National Cancer Center Singapore and the Sun Yat Sen University Cancer Centre. Overall 256 individuals (250 patients suffering from HCC or CHB, 6 healthy individuals) were included in this study. Except for healthy controls, all patients were positive to the surface antigen of the hepatitis B virus (HBsAg positive).

FIG. 2 shows the clinical characteristics of the study participants, with FIG. 2A showing in Table 1 the clinical characteristics of the patients recruited at Yat-sen University Cancer Center, GuangZhou and FIG. 2A showing in Table 2b the clinical characteristics of the patients recruited at the Singapore General Hospital and National Cancer Center Singapore. 75 patients from GuangZhou were diagnosed with HCC and 128 were Chronic Hepatitis patients while 35 patients from Singapore were diagnosed with HCC and 12 were Chronic Hepatitis patients.

FIG. 3 shows in Table 3 (FIG. 3A) the differential expression and diagnostic performance of the 9 significant genes identified in the training group of the present invention (Table 3). The training group (training set) included 50 patients diagnosed with HCC, and 50 patients diagnosed with chronic hepatitis. FIG. 3B shows the Area Under Curve (ROC) for the markers TNFAIP3 (curve (a)), the amphiregulin (AREG) gene (curve (b)), NFKB1A (curve (c)), NFKB1Z (curve (d)) and CD83 (curve (e)). FIG. 3C shows the ROC for the markers GTPase, IMAP family member 6 (GIMAP6) (curve (a)), GTPase IMAP family member 4 (GIMAP4) (curve (b)), GTPase IMAP family member 5 (GIMAP5) gene (curve (d)) and GTPase IMAP family member 8 (GIMAP8) (curve (e)). The Area Under Curve (AUC) in FIGS. 3A and 3B is shown with 95% confidence interval.

FIG. 4 shows ROC (receiver operating characteristic) curves analysis of different marker models in the training group (FIG. 4A) and the testing group (FIG. 4B) In more detail, the ROC curve analysis either for the tumor necrosis factor, alpha-induced protein 3 (TNFAIP3) gene alone (curve (a)) or the combination of TNFAIP3 together with the amphiregulin (AREG) gene and the GTPase IMAP family member 5 (GIMAP5) gene) (curve (b)) is shown. The Area Under Curve (AUC) in FIG. 4 is shown with 95% confidence Interval. The models were developed by stepwise logistic regression (forward method). The probability of being HCC was calculated from the odds ratio and was given as a score ranging from 0 to 1.

FIG. 5 shows the sensitivity (True Positive Rate (TPR)) and specificity (1-False Positive Rate (FPR)) from ROC analysis of training and testing groups for the TNFAIP3 gene alone or the combination of the TNFAIP3 gene together with the AREG gene and the GIMAP5 gene at different cutoff points between 55 and 92%.

FIG. 6 shows the ROC curves analysis of the TNFAIP3 gene alone (curve (a)) or the combination of the TNFAIP3 gene, the AREG gene and the GIMAP5 gene (curve (b)) and serum AFP (curve (c)) in 104 HCC and 108 CHB patients.

FIG. 7 shows the ROC curves analysis ROC curves analysis the TNFAIP3 gene alone (curve (a)) or the combination of TNFAIP3 gene, the AREG gene and the GIMAP5 gene (curve (b)) in comparison with serum AFP (curve (c)) in 14 patients that has been diagnosed with Barcelona Clinic Liver Cancer (BCLC) stage A HCC patients and 140 CHB patients.

FIG. 8 shows a Venn Diagram for three pairwise comparisons. Candidate gene markers were selected from those genes that are differentially expressed in HCC compared with CHB and healthy subjects (shaded area).

FIG. 9 shows the differential gene expression of the 9 genes that are significantly expressed in HCC and CHB as identified from the gene microarray analysis. These genes are TNFAIP3, AREG, GIMAP5, nuclear factor of kappa light polypeptide gene enhancer in B-cells inhibitor alpha (NFKBIA), nuclear factor of kappa light polypeptide gene enhancer in B-cells inhibitor zeta (NFKBIZ), CD83, GTPase IMAP family member 4 (GIMAP4), GTPase IMAP family member 6 (GIMAP6) and GTPase IMAP family member 8 (GIMAP8).

FIG. 10 shows the differential expression of the 9 WBC gene markers (TNFAIP3, AREG, GIMAP6, NFKBIA, NFKBIZ, CD83, GIMAP4, GIMAP5 and GIMAP8) in the training set validated by q-PCR (HCC patient number n=50; CHB patent number n=50; Healthy patient number n=6). Gene expression levels were normalized to that of CD45 (as reference gene) and presented as percentage of CD45 expression level. The box refers to the 25^{th} and 75^{th} percentile, with the line indicating the median. Whiskers represent the minimum and maximum values. Mann-Whitney test was performed to determine the significance.

FIG. 11 shows the primers used for the validation of the identified nine gene markers (TNFAIP3, AREG, GIMAP6, NFKBIA, NFKBIZ, CD83, GIMAP4, GIMAP5 and GIMAP8) by quantitative PCR.

### DETAILED DESCRIPTION OF THE INVENTION

The invention provides a sensitive and yet specific method of early diagnosis of liver cancer such as Hepatocellular carcinoma (HCC) at a point of time when patients do not show any symptoms of HCC. Thus the present application also provides a method that is able more accurately assess and stratify patients with different risks of disease occurrence or recurrence of liver cancer such as HCC. In addition, the present invention provides of method of assessing the risk of a subject having liver cirrhosis of developing liver cancer such as HCC and thus provides a significant clinical benefit compared to the currently used methods such as ultrasound or determination of serum α-fetoprotein (AFP) level. The methods of the inventions are thus extremely beneficial for the clinical management of HCC, including the risk management and monitoring of patients that are at risk or have a predisposition of developing advance HCC.

The invention is based on the finding that the immune system plays an important role at different stage of tumour development and that the appearance of tumour may lead to detectable gene expression patterns changes in leukocytes/white blood cells (WBC). Immune response-related gene signature has been identified in the nontumourous hepatic tissue in HCC patients to predict metastasis (Budhu et al, Cancer Cell (2006) August; 10(2):99-111. In the present invention, the inventors used a high density gene microarray to profile gene expression in the WBCs isolated from patients being infected with hepatitis B (HBV) and having HCC (HBV+HCC patients), patients having chronic hepatitis B ((CHB) patients) and healthy individuals.

In a first aspect, the invention is directed to a method of diagnosing liver cancer in a subject. This method comprises determining in a sample obtained from the subject the gene expression level of at least one marker gene selected from the group consisting of the tumor necrosis factor, alpha-induced protein 3 (TNFAIP3) gene, the amphiregulin gene and the GTPase, IMAP family member 5 (GIMAP5) gene.

The abbreviations TNFAIP3, AREG and GIMAP5 for the tumor necrosis factor, alpha-induced protein 3 gene, the amphiregulin gene and GTPase, IMAP family member 5 are the approved symbols from the HUGO Gene Nomenclature Committee (HGNC) database and are therefore used herein within their meaning as accepted and understood in the art.

The HGNC database identifier for the TNFAIP3 gene as used in the present invention is 11896, the Entrez Gene date base identifier is 7128. The TNFAIP3 gene as referred herein was identified as a gene whose expression is rapidly induced by the tumor necrosis factor (TNF). The protein encoded by this gene is a zinc finger protein with a length of 790 amino acids (UniProtKB accession number: TNAP3_HUMAN, Swiss-Prot accession number: P21580, SEQ ID NO: 19) and has been shown to inhibit NF-kappa B activation as well as TNF-mediated apoptosis. Knockout studies of a similar gene in mice suggested that this gene is critical for limiting inflammation by terminating TNF-induced NF-kappa B responses.

The HGNC database identifier for the AREG gene as used in the present invention is 651, the Entrez Gene date base identifier is 7128. The protein encoded by the AREG gene is also known as is a member of the epidermal growth factor family. The protein with a length of 252 amino acids (UniProtKB: AREG_HUMAN, Swiss Prot accession number P15514, SEQ ID NO: 20) is an autocrine growth factor as well as a mitogen for astrocytes, Schwann cells, and fibroblasts. It is related to epidermal growth factor (EGF) and transforming growth factor alpha (TGF-alpha). This protein interacts with the EGF/TGF-alpha receptor to promote the growth of normal epithelial cells and inhibits the growth of certain aggressive carcinoma cell lines. This encoded protein is associated with a psoriasis-like skin phenotype.

The HGNC database identifier for the GIMAP5 gene as used in the present invention is 18005, the Entrez Gene date base identifier is 55340. The GIMAP5 gene encodes a protein with a length of 408 amino acids (UniProtKB: Q96F15, Swiss Prot accession number: Q96F15, SEQ ID NO: 21) belonging to the GTP-binding superfamily and to the immuno-associated nucleotide (IAN) subfamily of nucleotide-binding proteins. In humans, IAN subfamily genes are located in a cluster at 7q36.1. Two transcript variants, one protein-coding (Q96F15-1) and the other probably non-protein-coding (Q96F15-2), have been found for this gene. The use of both transcripts (gene variants) is within the scope of the present invention.

One embodiment of this method of detection liver cancer comprises determining the expression level of at least two of the marker genes selected from the group consisting of the TNFAIP3 gene, the AREG gene and the GIMAP5 gene, that means the expression level of a) the TNFAIP3 gene and the AREG gene, or b) the TNFAIP3 gene and the GIMAP5 gene, or c) the AREG gene and the GIMAP5 gene together. In a further embodiment, the method comprises determining the expression level of all three of the TNFAIP3 gene, the AREG gene and the GIMAP5 gene.

In addition to any of these three marker genes, a method of detecting liver cancer can comprise the detection of one of more of the following 6 markers: nuclear factor of kappa light polypeptide gene enhancer in B-cells inhibitor alpha (NFKBIA), nuclear factor of kappa light polypeptide gene enhancer in B-cells inhibitor zeta (NFKBIZ), CD83, GTPase IMAP family member 4 (GIMAP4), GTPase IMAP family member 6 (GIMAP6) and GTPase IMAP family member 8 (GIMAP8). In these embodiments, either one, two or all three of the TNFAIP3 gene, the AREG gene and the GIMAP5 gene can be used together with one, two, three, four, five or all these six marker genes selected from the group of NFKBIA, NFKBIZ, CD83, GIMAP4, GIMAP5 and GIMAP8. In this respect, it is noted that the present invention also encompasses the sole use of any of the marker genes for diagnosing liver cancer such as HCC selected from the group consisting of nuclear factor of kappa light polypeptide gene enhancer in B-cells inhibitor alpha (NFKBIA), nuclear factor of kappa light polypeptide gene enhancer in B-cells inhibitor zeta (NFKBIZ), CD83, GTPase IMAP family member 4 (GIMAP4), GTPase IMAP family member 6(GIMAP6) and GTPase IMAP family member 8 (GIMAP8).

In this respect, it is noted that the expression level of a gene of interest can either be down-regulated or up-regulated. It has for example been found in the present invention that five genes used in the present invention (TNFAIP3, AREG, NFKBIA, NFKBIZ, CD83) have higher expression levels in HCC than that in a control while the four genes from the GIMAP family (GIMAP4, GIMAP5, GIMAP6 and GIMAP8) that are used in the present invention have lower expression levels in HCC than of a control. The term “determining the expression level” as used herein usually refers to the determination of the amount of the respective mRNA of the gene of interest in a sample that is obtained from a subject. The expression level can be determined using any methodology that is available and well known to the person skilled in the art. For example, the mRNA can be isolated from a sample of a subject, and the reversely described into cDNA using commercially available kits such as but not limited to the SuperScript® III First-Strand Synthesis System (Invitrogen, USA). Therefore, the cDNA so obtained (or a part thereof) can be assayed by nucleic acid amplification methods such as, but not limited, to real-time PCR, quantitative PCR, isothermal nucleic acid amplification, or ligase chain reaction (LCR) to name only a few.

Determining the expression level may include using a reference gene that this constitutively expressed in a sample of the subject. The determination may also include comparing the expression level to a control sample that expresses the gene of interest. The determination of the expression level may be carried out qualitatively, that means, only the presence or absence of a gene product might be determined, or quantitatively, that means the total amount of the expression product (relative to a control sample) might be determined.

A method of the invention can be used to diagnose any form of liver cancer (hepatic cancer) that originates in the liver. By “liver cancer” is meant a malignant tumour that grows on the surface or inside the liver. The liver cancer can for example be hepatocellular carcinoma (HCC) or a variant type thereof that consists of both HCC and cholangiocarcinoma (bile duct cancer) components. The liver cancer can also be sarcoma, hepatoblastoma or cancer of the mesenchymal tissue.

A diagnosis method as disclosed herein can be applied to any subject, typically any mammal including human. One significant advantage of a method of the invention is that it is sensitive and specific even if at an early stage of disease development when the subject/human does not shown signs of liver cancer such as HCC. Accordingly, the present invention also constitutes a significant advantage in patient management since it allows the monitoring and also potential treatment of a patient at a time when the patient is still asymptotic. Thus, the present invention allows the monitoring of patients that are at high risk of developing HCC such as patients suffering from chronic hepatitis B (CHB) or patient suffering from both chronic hepatitis B and liver cirrhosis. Since far surgery of early HCC is the only cure, the present application thus also allows recognizing the occurrence/development of HCC at a very early stage and thus to increase the survival and curing rate of HCC patients. The method of measuring either the gene expression level of at least one of the tumor necrosis factor, alpha-induced protein 3 (TNFAIP3) gene, the amphiregulin (AREG) gene and the GTPase IMAP family member 5 (GIMAP5) gene or of measuring the presence or amount of at least one of these three proteins (TNFAIP3, AREG or GIMAP5) (which is explained in detail below) can also be used to monitor patients that have undergone surgical treatment to check for the re-occurrence of HCC.

As mentioned above, in a method of the invention the determined expression level in a sample obtained from a subject may be compared to a control sample. An increased expression level in the sample of the subject/patient of interest relative to the control sample can thus be indicative of a risk of developing liver cancer such as HCC.

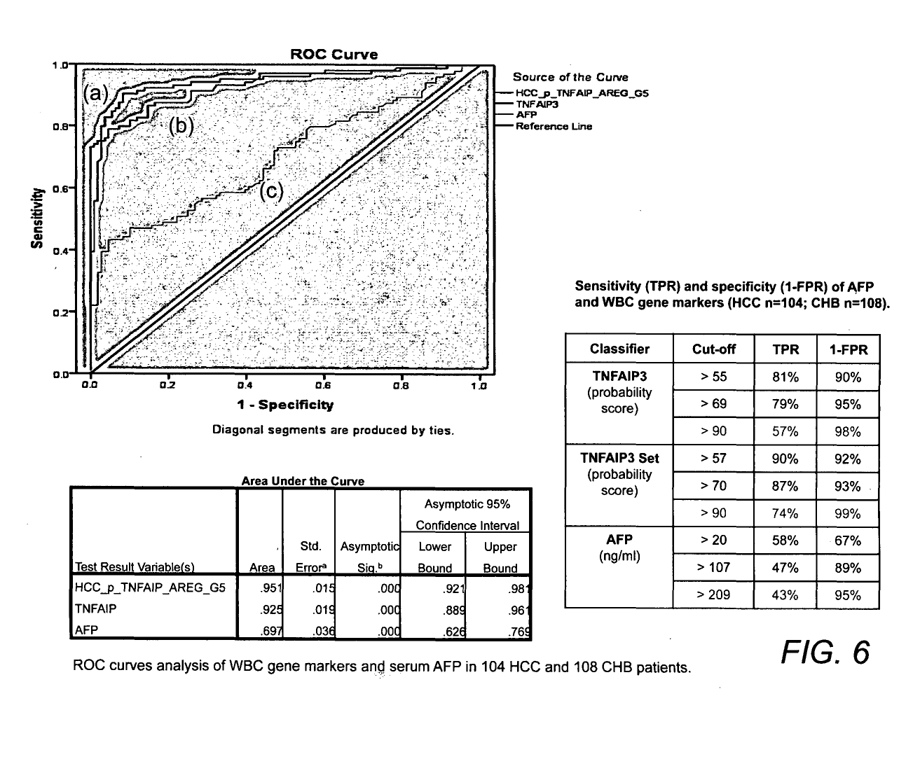

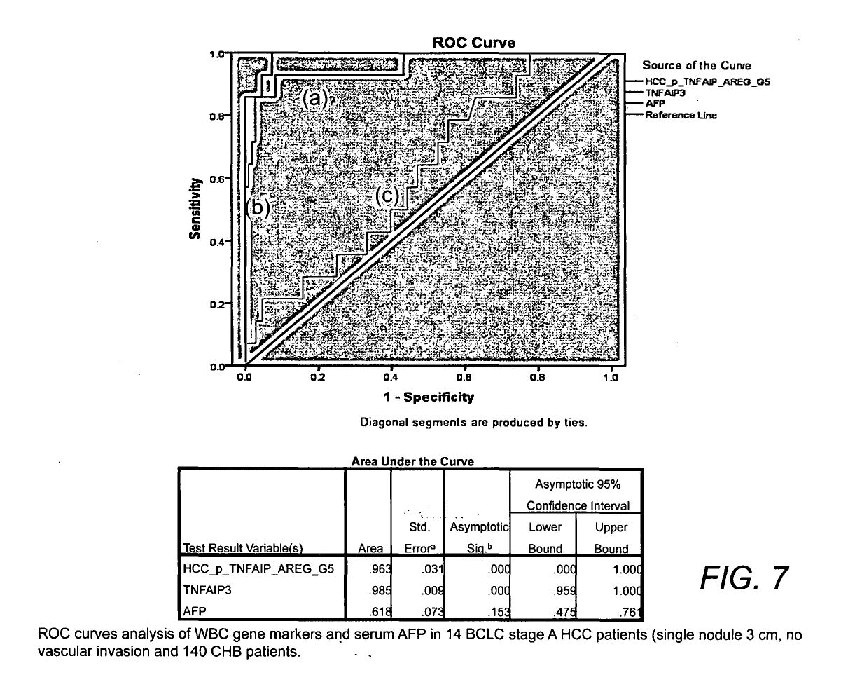

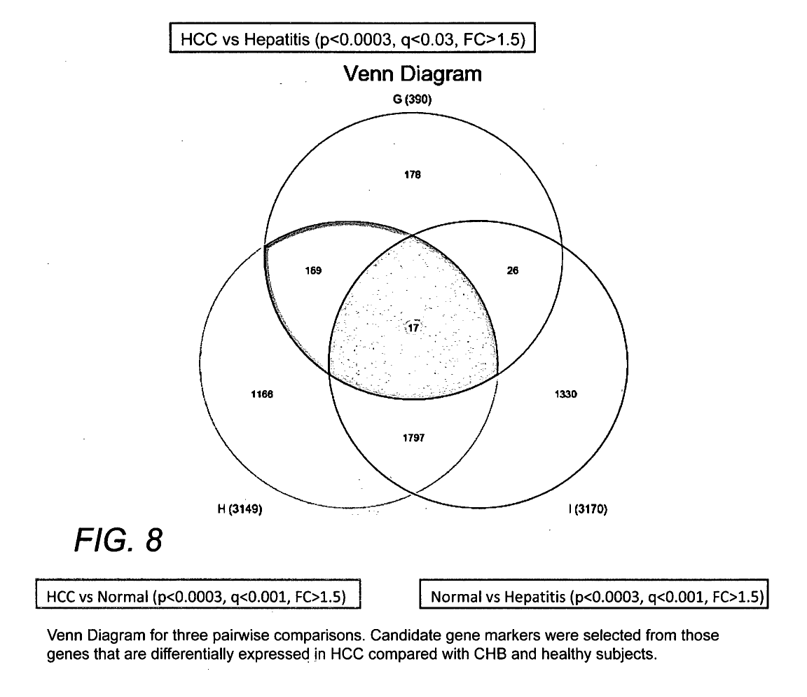

A further advantage of a method of the present invention is that it allows distinguishing a subject suffering from HCC or having a risk of developing HCC from a subject suffering from Chronic Hepatitis B (see experimental section, FIGS. 6 and 7). At present, this differentiation is very difficult to make. The HCC that is distinguished at that time might be Barcelona Clinic Liver Cancer (BCLC) stage A HCC. The method of the invention also allows the risk assessment or diagnosis of patients that suffer from liver cirrhosis and thus allows to determine whether the liver cirrhosis is associated with liver cancer such as HCC or, for example, Hepatitis B. The ability to distinguish these two patient groups is another significant advantage of the present invention.

A diagnosis as described herein can be carried out with any suitable body or tissue sample from the patient, including solid samples such as tissue or body fluids. The sample may advantageously comprises or be a blood cell such as a peripheral blood mononuclear cell (PBMC) or liver tissue. The blood cell is typically a leucocyte.

In line with the above, the invention also provides a method of assessing the risk of a subject having liver cirrhosis of developing liver cancer. This method comprises determining in a sample obtained from the subject the gene expression level of at least one marker gene selected from the group consisting of the tumor necrosis factor, alpha-induced protein 3 (TNFAIP3) gene, the amphiregulin (AREG) gene and the GTPase, IMAP family member 5 (GIMAP5) gene. Also in this aspect the liver cancer may be hepatocellular carcinoma (HCC) and the subject/patient may not show signs of HCC at the time of testing.

As an alternative to determining the expression level of one or more genes that have been identified here as markers for diagnosing of liver cancer in a subject, the present invention also encompasses determining in the sample obtained from a subject the presence or amount of at least one marker protein that is encoded by one of the genes identified here. Thus, the invention is also directed to determining the presence of at least one marker protein selected from the group consisting of tumor necrosis factor, alpha-induced protein 3 (TNFAIP3, SwissProt accession number: P21580), amphiregulin (AREG, SwissProt accession number P15514:) and GTPase, IMAP family member 5 (GIMAP5, SwissProt accession number Q96F15). In embodiments of this method, the presence or amount of two of these three or the presence or amount of all three proteins is determined. This method can be used for diagnosis of any of the liver cancers mentioned above with HCC being the most preferred cancer.

Other embodiments of this method of diagnosing liver cancer may comprise the determination of the presence or amount of one of more of the following 6 markers proteins: nuclear factor of kappa light polypeptide gene enhancer in B-cells inhibitor alpha (NFKBIA), nuclear factor of kappa light polypeptide gene enhancer in B-cells inhibitor zeta (NFKBIZ), CD83, GTPase IMAP family member 4 (GIMAP4), GTPase IMAP family member 6 (GIMAP6) and GTPase IMAP family member 8 (GIMAP8). In these embodiments, either one, two or all three of the TNFAIP3 gene, the AREG gene and the GIMAP5 gene can be used together with one, two, three, four, or all these five marker genes selected from the group of NFKBIA, (NFKBIZ), CD83, GIMAP4, GIMAP6 and GIMAP8. In this respect, it is noted that the present invention also encompasses the sole use of any of the marker genes for diagnosing liver cancer such as HCC selected from the group consisting of nuclear factor of kappa light polypeptide gene enhancer in B-cells inhibitor alpha (NFKBIA), nuclear factor of kappa light polypeptide gene enhancer in B-cells inhibitor zeta (NFKBIZ), CD83, GTPase IMAP family member 4 (GIMAP4), GTPase IMAP family member 6 (GIMAP6) and GTPase IMAP family member 8 (GIMAP8).

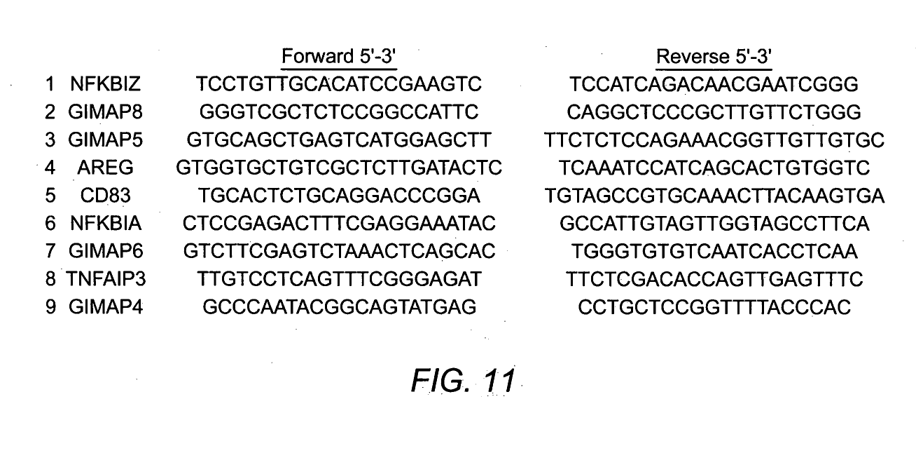

The present invention is also directed to a kit for the diagnosis of liver cancer by determining the expression level of at least one marker gene selected from the group consisting of the alpha-induced protein 3 (TNFAIP3) gene, the amphiregulin (AREG) gene and the GTPase, IMAP family member 5 (GIMAP5) gene. The kit comprises one or more oligonucleotides complementary to at least one of the marker gene nucleic acid molecule. The kit may comprise two kinds of one or more oligonucleotides, wherein each kind of oligonucleotide is complementary to one of at least two of the marker gene nucleic acid molecules. The kit may also comprise three kinds of one or more oligonucleotides, wherein each kind of oligonucleotide is complementary to one at of the three marker gene nucleic acid molecules. (cf. the Experimental Section or FIG. 11 showing suitable oligonucleotides for the amplification and quantification of the nine gene markers identified herein). The oligonucleotides are usually oligonucleotide probes such as amplification primers/probes which, for example, can be used for the amplification of the respective marker gene after transcription of the isolated total mRNA from the sample of the subject to be examined. Thus, such amplification primers are suitable to amplify a marker nucleic acid molecule in an amplification step. Since the markers genes identified in the present invention as known as such, the design of suitable amplification primers is within the knowledge of the person of average skill in the art. The oligonucleotides used in the kit can be of any length, for example, can be up to about 30, about 60, or about 100 nucleotides in length. These oligonucleotides (probes) can also be labelled, for example to allow real-time PCR or quantification of the marker gene of interest. The label might for example be a radioactive label, a fluorescent label, a chemiluminescent label, an affinity label (for example, for immobilising the oligonucleotide on a solid phase in a heterogeneous assay format) or an enzymatic label. The affinity label may be reagent that is commonly used in the detection of nucleic acids. Examples of such as reagent include, but are not limited to biotin or digoxigenin. The gene expression (level) can be determined by any suitable methodology available and can, for example, be carried out using commercially available systems such as the Affymetrix QuantiGene Plex 2.0 (Affymetrix, Santa Clara, Calif., USA) which are commonly used for testing, validation and quantification of disease biomarkers. With such assays, it is currently possible to analyse the gene expressing of 3 to 80 marker genes simultaneously by multiplexing. In brief, in such assays, a tissue or body sample (e.g. a PBMC) is lysed to release the RNA and contacted with solid supports such as magnetic beads on which a panel of probes specific to the genes of interest are immobilized. The purified RNA sample is incubated over a suitable period of time such as 24 hours for hybridization of the respective probes with the RNA of the marker gene of interest. After target hybridization, signal amplification is achieved using branch DNA (bDNA) technology (see for example, the product description of the QuantiGene Plex 2.0 for details). Finally, a detection compound that generates a signal that is proportional with the amount of target RNA present in the sample is added and the optical signal is read using a respective reader such as a luminescence or fluorescence reader.

If the presence or amount of one or more of the marker proteins identified herein is to be determined, the determination is carried out with any assay method configured to detect the one or more proteins in a sample such as a tissue or body fluid sample obtained from the subject to provide an assay result. The assay might be am immunoassay such an ELISA (for which polyclonal or monoclonal antibodies against the protein of interest, e.g. TNFAIP3, the amphiregulin AREG and the GTPase IMAP family member 5 (GIMAP5) can be used). In general, immunoassays involve contacting a sample containing or suspected of containing a protein (marker) of interest with at least one antibody that specifically binds to the protein (marker). A signal is then generated indicative of the presence or amount of complexes formed by the binding of polypeptides in the sample to the antibody. The signal is then related to the presence or amount of the biomarker in the sample. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994, each of which is hereby incorporated by reference in its entirety, including all tables, figures and claims.

The assay devices and methods known in the art can utilize labeled molecules in various sandwich, competitive, or non-competitive assay formats, to generate a signal that is related to the presence or amount of the protein of interest, that means here, at least one of the TNFAIP3, AREG and GIMAP5. Both monoclonal and polyclonal antibodies against TNFAIP3, the amphiregulin AREG and the GTPase IMAP family member 5 (GIMAP5) are commercially available from a variety of sources. See, an purely illustrative examples, Proteintech Group, Inc. (Chicago, Ill., USA) polyclonal TNFAIP3 rabbit antibody Catalog No.: 23456-1-AP, Pierce (Thermo Fisher Scientific, Rockland, Ill., USA) Amphiregulin Polyclonal Antibody catalogue number PA5-16616, Santa Cruz Biotechnology Inc. (Santa Cruz, Calif., USA), monoclonal mouse GIMAP5 Antibody (E-11), catalogue number sc-377307. Alternatively, such antibodies can be obtained by immunization or from artificial antibody library using recombinant antibody engineering techniques (evolutionary methods) such as phage display.

The presence or amount of the protein of interest may also be determined by means other than immunoassays, including protein measurements (such as dot blots, western blots, chromatographic methods, mass spectrometry, etc.).

### Examples

### Materials and Methods

### Patients

Patients with primary HCC were recruited at diagnosis at National Cancer Centre Singapore (NCCS). Some HCC blood samples were also collected at the Department of Hepatobiliary Oncology, Sun Yat-Sen University Cancer Center. Blood samples of patients with chronic hepatitis and cirrhosis (CHB) and HCC were also recruited at varying times during their visits to the clinic at the Department of Gastroenterology, Singapore General Hospital. All samples were collected according to the protocols approved by the respective Institutional Review Board and informed consent was obtained from all subjects before blood samples were collected. All healthy participants were staff of NCCS who have no history of liver disease, no viral hepatitis, and no malignant disease and blood samples were collected after verbal informed consent. A total of 10 ml of blood was collected into BD Vacutainer® Plus Plastic K2 EDTA tubes (Becton-Dickinson).

Diagnosis for HCC was made either by histological evaluation or two dynamic imaging examination, according to AASLD guidelines (Bruix & Sherman, 2011, supra). Blood samples were collected before any treatment was given. Patients with any comorbidity were excluded.

For chronic hepatitis B (CHB) patients, AASLD Practice Guidelines were used as inclusion criteria, which include HBsAg positive >6 months; HBV DNA >10³ copies/ml (10⁴-10⁵ copies/ml for HBeAg negative cases), and persistent or intermittent aspartate aminotransferase/alanine aminotransferase (ALT/AST) elevation in serum. Patients with any comorbidity were excluded.

The diagnosis of cirrhosis was based on imaging evidence and has had no evidence of a hepatic mass for at least 3 months before enrolment.

Liver Samples

Cancerous and the corresponding distal non-cancerous liver tissues were obtained from patients who underwent partial hepatectomy as curative treatment for HCC. All cancerous tissues studied were at least 70% cancerous. All tissue samples employed in this study were approved and provided by the Tissue Repository of the National Cancer Centre Singapore (NCCS), in accordance with the policies of its Ethics Committee. Informed consent was obtained from all participating patients and all clinical and histopathological data provided to the researchers were rendered anonymous.

### White Blood Cell Isolation

Within 6 hours of collection, blood was processed by density gradient centrifugation using Ficoll-Paque PLUS (GE Healthcare). Ficoll-Paque PLUS is an aqueous solution of density 1.077+0.001 g/ml containing 5.7 g Ficoll 400 and 9 g sodium diatrizoate with 0.0231 g calcium disodium ethylenediamintetraacetic acid in every 100 ml. Residual red blood cells were lysed in 1 ml RBC lysis buffer (BioLegend) for 5 min, and then washed with 10 ml phosphate buffered saline. The isolated PBMC were stored at −80° C. until testing. The clinical characteristics of the patients whose PBMC samples were studied are summarized in Tables 1 and 2.

RNA Extraction and Affymetrix Gene Chip Analysis

Total RNA was extracted from WBC using TRIzol reagent (Invitrogen, USA) and was quantified on an ND-1000 Nano-drop Spectrophotometer (Thermo Scientific, USA). The integrity of RNA was assessed by Agilent 2100 Bioanalyzer (Agilent, USA). Only those RNA samples with a RNA Integrity Number (RIN) greater than 6.7 were used for gene microarray as previously described in “Synthesis of Biotin-Labeled RNA for Gene Expression Measurements Using Oligonucleotide Arrays”. Ana E. Vázquez, Liping Nie, and Ebenezer N. Yamoah. Methods Mol. Biol. 2009; 493: 21. The final cRNA obtained was hybridized to the GeneChip Human Genome U133 Plus 2.0 Array (Affymetrix, USA), as described previously (Wang S M, Ooi L L, Hui K M. Identification and validation of a novel gene signature associated with the recurrence of human hepatocellular carcinoma. Clin Cancer Res 2007; 13:6275-83, Liu B H, Goh C H, Ooi L L, Hui K M. Oncogene. 2008 Jul. 3; 27(29):4128-36 “Identification of unique and common low abundance tumour-specific transcripts by suppression subtractive hybridization and oligonucleotide probe array analysis”. All data generated by the Affymetrix Microarray Suite version 5.0 in cel file format were refined using the Partek Genomics Suite software package (Partek, USA).

Quantitative PCR and Multiplexed Gene Expression Analysis

Quantitative PCR (q-PCR) was performed to validate the 9 candidate genes identified from gene microarray. The primers used for the amplification of the identified genes are depicted in FIG. 11 and are also given in the following table.

| | Forward 5′-3′ | Reverse 5′-3′ |
|---|---|---|
| NFKBIZ | TCCTGTTGCACATCCGAAGTC | TCCATCAGACAACGAATCGGG |
| | (SEQ ID NO: 1) | (SEQ ID NO: 2) |
| GIMAP8 | GGGTCGCTCTCCGGCCATTC | CAGGCTCCCGCTTGTTCTGGG |
| | (SEQ ID NO: 3) | (SEQ ID NO: 4) |
| GIMAP5 | GTGCAGCTGAGTCATGGAGCTT | TTCTCTCCAGAAACGGTTGTTGTGC |
| | (SEQ ID NO: 5) | (SEQ ID NO: 6) |
| AREG | GTGGTGCTGTCGCTCTTGATACTC | TCAAATCCATCAGCACTGTGGTC |
| | (SEQ ID NO: 7) | (SEQ ID NO: 8) |
| CD83 | TGCACTCTGCAGGACCCGGA | TGTAGCCGTGCAAACTTACAAGTGA |
| | (SEQ ID NO: 9) | (SEQ ID NO: 10) |
| NFKBIA | CTCCGAGACTTTCGAGGAAATAC | GCCATTGTAGTTGGTAGCCTTCA |
| | (SEQ ID NO: 11) | (SEQ ID NO: 12) |
| GIMAP6 | GTCTTCGAGTCTAAACTCAGCAC | TGGGTGTGTCAATCACCTCAA |
| | (SEQ ID NO: 13) | (SEQ ID NO: 14) |
| TNFAIP3 | TTGTCCTCAGTTTCGGGAGAT | TTCTCGACACCAGTTGAGTTTC |
| | (SEQ ID NO: 15) | (SEQ ID NO: 16) |
| GIMAP4 | GCCCAATACGGCAGTATGAG | CCTGCTCCGGTTTTACCCAC |
| | (SEQ ID NO: 17) | (SEQ ID NO: 18) |

Five hundred nanogram total RNA was reverse transcribed into cDNA using SuperScript® III First-Strand Synthesis System (Invitrogen, USA), and one fortieth of the cDNA was subsequently assayed by real-time PCR using SsoFast EvaGreen Supermix (Bio-Rad, USA). Comparative cycle threshold (Ct) method was used, and the expression levels of candidate genes were normalized to that of CD45, and −ΔΔCt was used in subsequent analysis. The efficiency of PRC reactions for candidate genes and reference gene were tested to be >90%.

Statistical Analysis

Serum AFP is the most commonly used serological marker for HCC screening and diagnosis, with an overall sensitivity of 52% and specificity of 80% (Daniele et al, Gastroenterology. 2004, supra)). In the training set that used a smaller group of 50 HCC and 50 CHB patients, the genes marker that were identified showed greater than 92% sensitivity and greater than 96% specificity. Hence, the training set/study was designed to compare the sensitivity of the identified gene markers to that of AFP to differentiate HCC from CHB patients. A sample size of 109 patients (50 HCC and 59 CHB) was required to achieve 90% power with 5% one-sided type I error (cf. Sample Size Tables for Clinical Studies, 3rd Edition, David Machin, Michael J. Campbell, Say-eng Tan, Sze-Huey Tan, ISBN: 978-1-4051-4650-0.) The software “Sample Size Tables for Clinical Studies Software Program Version 1.0” was used for the analysis.

Data obtained from the training sample set by quantitative polymerase chain reaction was used to construct a model using stepwise forward method logistic regression. The probability of being HCC was calculated using the logistic regression model and was given as a score ranging from 0 to 1. The HCC probability scores were used to generate receiver operating characteristic (ROC) curves. Area under the curve (AUC) was calculated. Sensitivity and specificity at different cutoff points were selected from the ROC curve from the training sample set.

The program PASW® Statistics 18 (SPSS Inc., Chicago, Ill., USA) was used for generating ROC curve, logistic regression, and statistical analysis. The Student's t-test or Mann-Whitney U test was used for the comparison of continuous variables, and the Chi-square test was used for categorical variables. Confidence interval of 95% was given for AUC, sensitivity and specificity.

Results Patient Characteristics

Patients with HCC or CHB were recruited at four different hospitals. Their characteristic information is listed in (Table 1 and 2).

Selection of Candidate WBC Gene Markers Using Gene Microarray

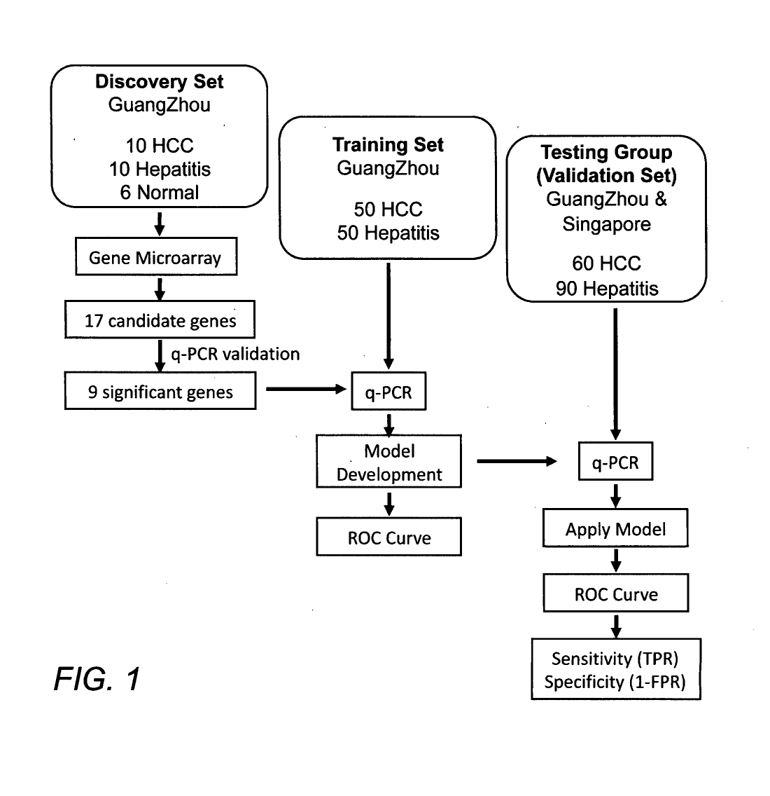

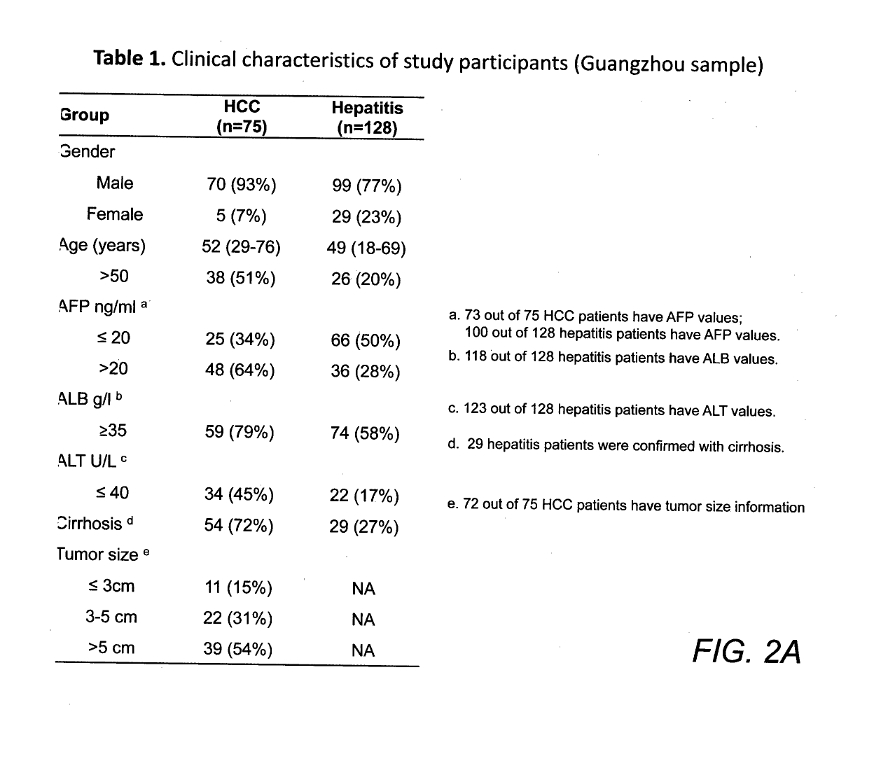

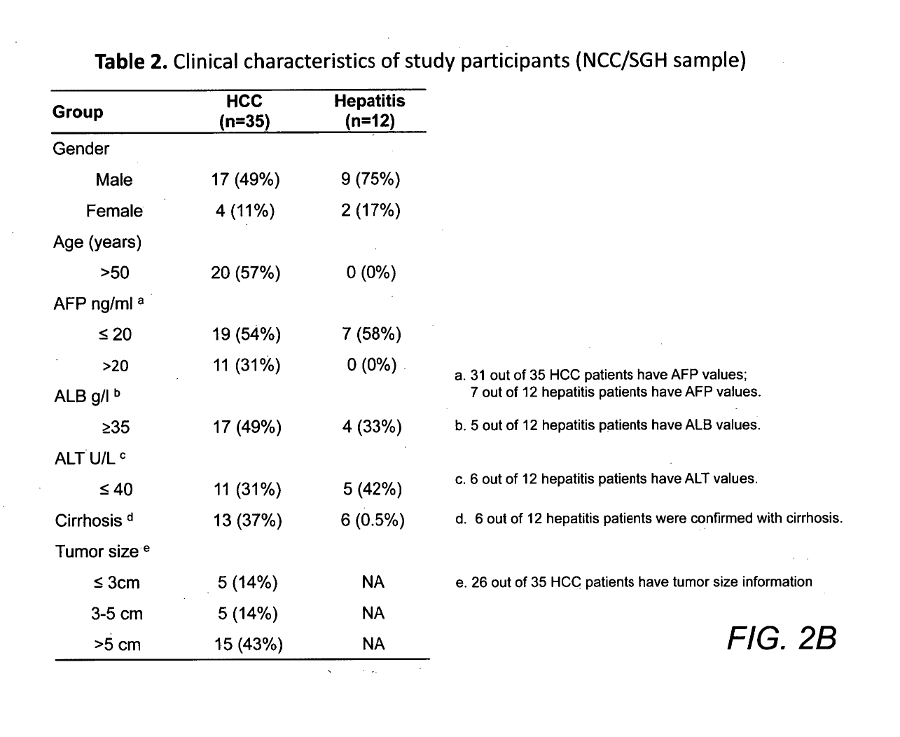

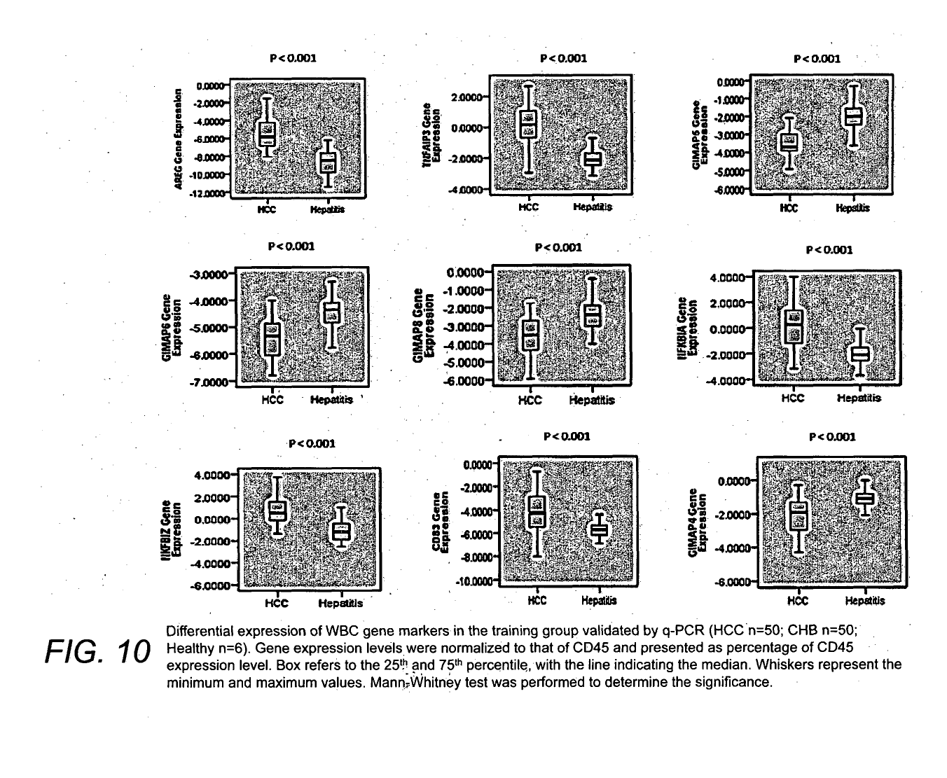

The high-density Affymetrix GeneChip Human Genome U133 Plus2.0 arrays were used to screen for potential gene markers from peripheral blood WBC. Total RNA extract from 28 samples (10 HCC, 12 CHB and 6 healthy patients) were used in this initial screening step (cf. FIG. 1). Candidate gene markers were selected from those genes that are differentially expressed in HCC compared with CHB and healthy subjects (FIG. 10). Several factors were taken into consideration in selecting candidate gene markers: fold change (>1.5 for up-regulated genes, <−1.5 for down-regulated genes), p-value (<0.0003), According these criteria, 9 genes were eventually selected for further validation by quantitative PCR (qPCR).

Validation of Candidate WBC Gene Expression by q-PCR

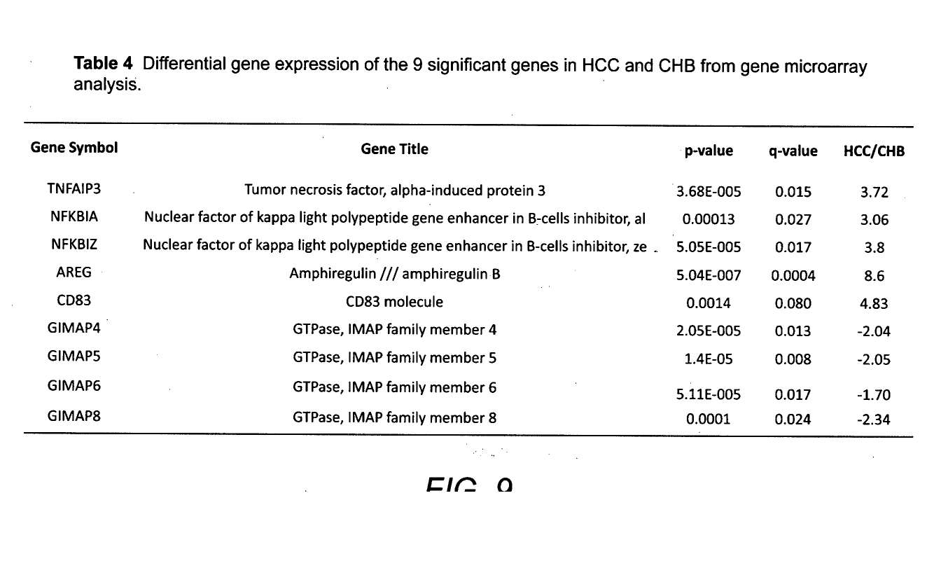

The expression levels of the 17 candidate genes were evaluated by q-PCR in a group consisting of 56 samples, which includes the 26 samples used in microarray screening and an additional 30 samples (15 HCC and 15 CHB). Among the 17 candidate genes, 9 genes showed significantly different expression level in HCC compared with CHB and healthy subjects (FIG. 10). ROC curve analysis of the 5 up-regulated and 4 down-regulated genes is shown in Table 2. All 10 predictors have an AUC greater than 0.7, while TNFAIP3 is the most powerful predictor (AUC 0.943). Five genes (TNFAIP3, AREG, NFKBIA, NFKBIZ, CD83) have higher expression levels in HCC than that in control, with fold change ranging from 2.8 to 8.2. Four genes from GIMAP family (GIMAP4, GIMAP5, GIMAP6 and GIMAP8) have lower expression levels in HCC than that in control, with a lesser degree fold change ranging from 1.9 to 2.4 (FIG. 9). The qPCR data of these 9 significant genes in this group were used as a training data set to develop a model to combine the discriminating power of the individual genes.

Model Development and WBC Gene Marker Selection

Since some of the significant genes are in the same signaling pathway, or in the same gene family, their gene expression could be correlated with each other. Hence, a multicollinearity test was applied before the gene expression data were used for model development. The test showed that the Variance Inflation Factor index (VIF) of TNFAIP3, NFKBIA and NFKBIZ are greater than 5, indicating multicollinearity is present Since TNFAIP3 is the most powerful predictor, NFKBIA and NFKBIZ were removed from the panel. Multicollinearity test was applied again and no multicollinearity was detected with the remaining 8 predictors with VIF less than 5. A stepwise logistic regression model was developed using the forward method:

Log(p/(1+p))=3.462+0.897×AREG+1.570×TNFAIP3−1.769×GIMAP5

TNFAIP3, AREG and GIMAP5 were included in the model as independent predictors, while the other five genes appeared to be redundant. According to the model, the probability of being HCC in a certain subject was calculated. Similarly, a logistic regression was done based on a single predictor TNFAIP3 as a comparison (Log(p/(1+p))=2.812+2.403×TNFAIP3), and the probability score was calculated.

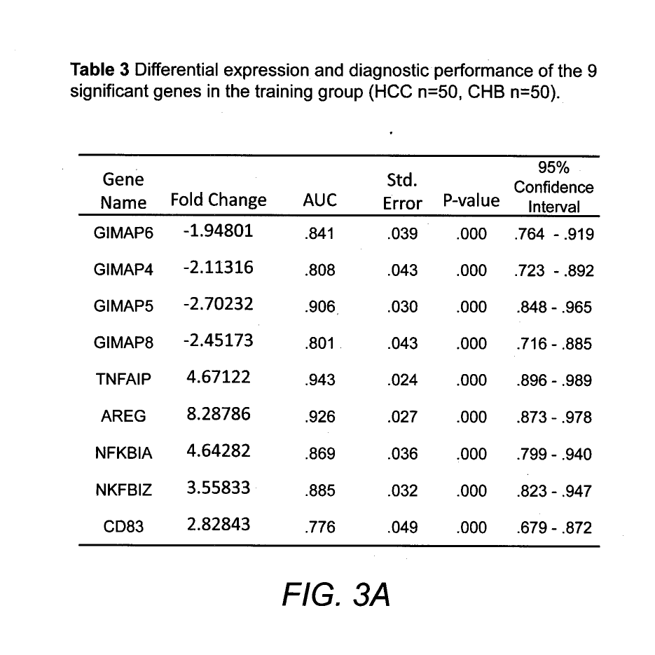

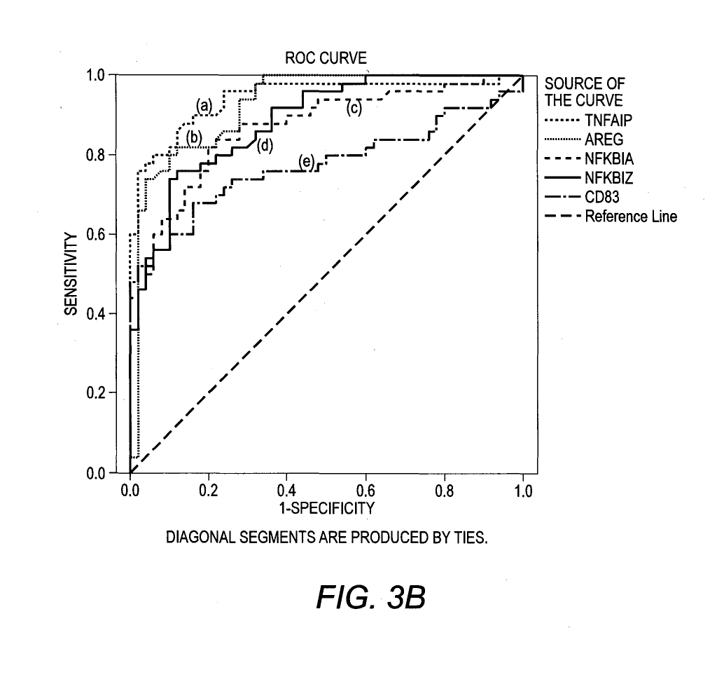

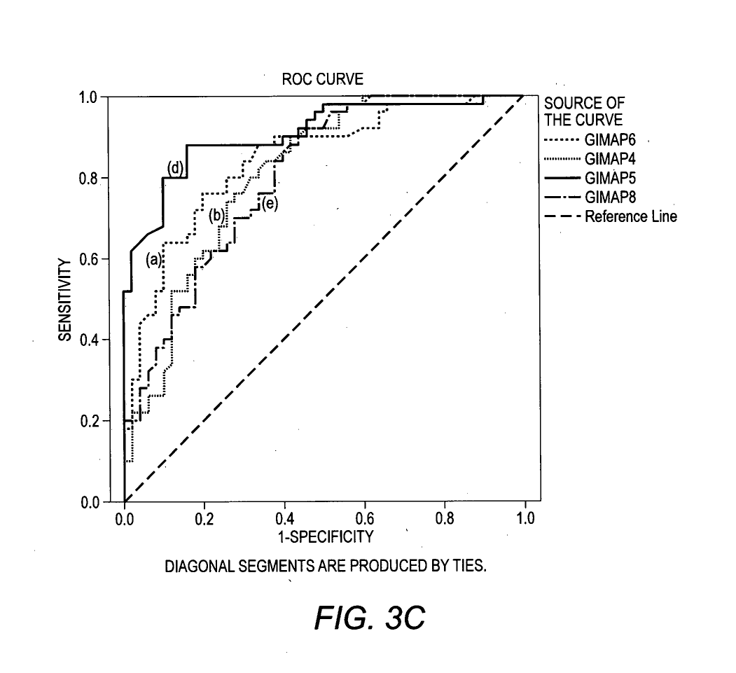

The probability scores from both models were used to generate ROC curves for the training group (FIG. 3A). Both the single gene and 3-gene model are excellent predictors (AUC>0.9), while the 3-gene model further increased the AUC to 0.977.

Validation of the WBC Gene Marker Panel

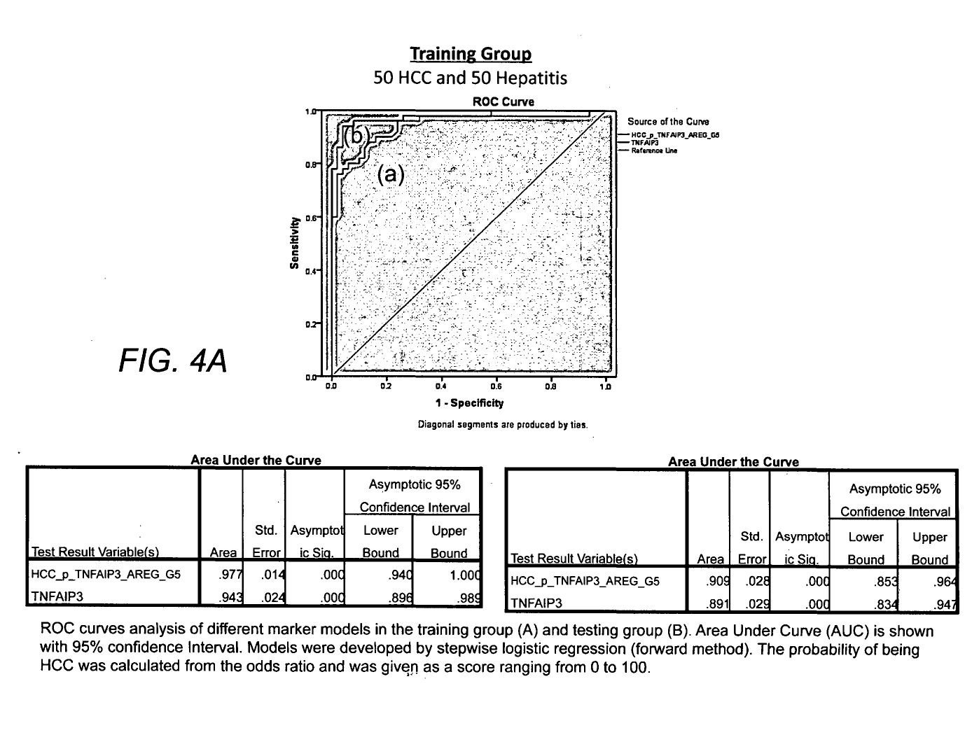

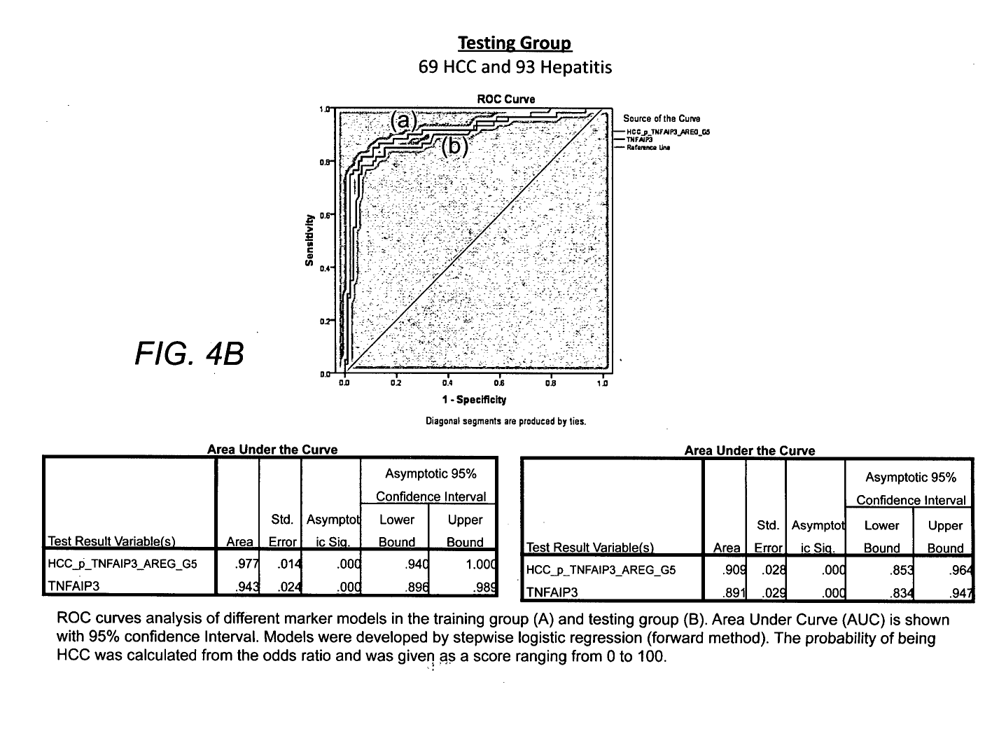

The models developed in the training group were validated in an independent sample group of 60 HCC and 90 CHB patients. The ROC curves are shown in FIG. 4B. Compared with that in the training group, AUC in the testing group decreased slightly to 0.891 for single gene and 0.909 for the 3-gene model.

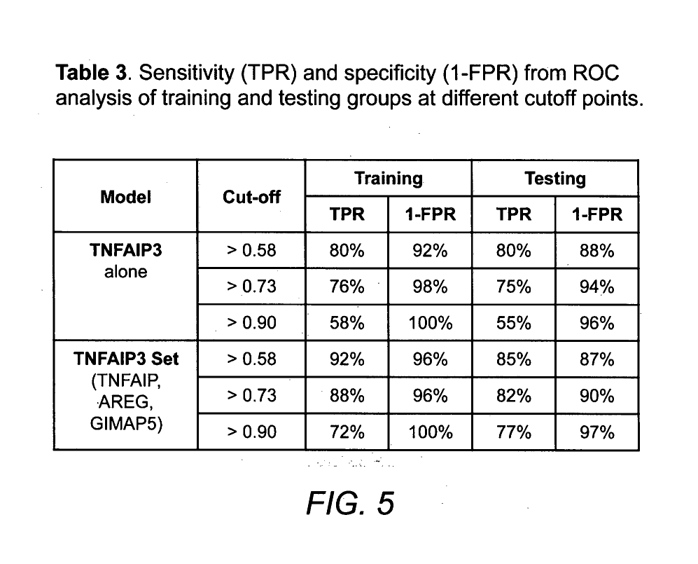

Accordingly, the sensitivity and specificity in both the training and testing group at different cut-off points are listed in Table 3 (FIG. 5). At the same cut-off point, the sum of sensitivity and specificity in the training group is higher than that in the testing group for both single gene and 3-gene models. At a lower cut-off point of HCC probability score (ranging from 55 to 70), the two models perform similarly in distinguishing HCC from CHB. However, at a higher cut-off point around 90, a higher sensitivity of 72% was achieved by the 3-gene model than that by the single gene model which gave a sensitivity of 58%, while the specificity is 100% for both models.

In addition, because serum AFP is the most commonly used serological marker for HCC diagnosis and screening, its ability to detect HCC from CHB was compared with that of the WBC gene markers of the present invention, TNFAIP3, AREG and GIMAP5. A total of 104 HCC and 108 CHB patients with available AFP data were used in this comparison. The ROC curve analysis shows that both the single gene and 3-gene model perform significantly better than AFP (FIG. 6). While the AUC for AFP is 0.697, the AUC for the WBC gene markers are both more than 0.94. At a cut-off point of 200 ng/ml AFP for clinical diagnosis (Bruix & Sherman, 2011, supra), the sensitivity is 43% and the specificity is 95%. In contrast, the sensitivity if 74% and specificity is 99% for the 3-gene model, and slighter lower for the single gene model (sensitivity 57%, specificity 98%).

Furthermore, both AFP and the gene markers of the present invention, TNFAIP3, AREG and GIMAP5 were applied to distinguish patients with Barcelona Clinic Liver Cancer (BCLC) stage A HCC (single nodule less than or ≦3 cm, no vascular invasion) from CHB patients. Similar to the result from all HCC patient group, the gene markers of the present invention perform better than AFP with AUC greater than 0.96 (FIG. 7).

Discussion

Currently, AFP is the most commonly used serological marker for HCC, with unsatisfactory sensitivity and specificity. Due to its inadequate accuracy, the American Association for the Study of Liver Diseases (AASLD) practice guidelines published in 2010 no longer recommend AFP as a marker for HCC screening and diagnosis (Bruix & Sherman 2011, supra).

The present invention aimed to discover effective new markers from peripheral blood to detect HCC at early stage. According to a 5-phase structure used by the Early Detection Research Network (EDRN) of U.S. National Cancer Institute, this is a phase 2 study for clinical assay development and validation. Patients with chronic hepatitis B and patients with HBV-associated HCC were recruited as most HCC is developed in HBV positive population in Asia. By using comprehensive gene expression profiling microarray, candidate gene markers were identified in WBC from HCC patients. q-PCR was used to validate the candidate genes, and subsequently for measuring gene expression levels in clinical sample for its simplicity and reproducibility.

9 genes from 17 candidate genes were validated in the present invention by q-PCR, and used a training group to develop a logistic model which comprised three genes. The 3-gene model has excellent diagnostic accuracy in both the training and independent testing group (FIG. 7). Even though TNFAIP3 alone can achieve 80% sensitivity and 88% specificity, the 3-gene model can fine tune the accuracy and resulted in a higher sensitivity when high specificity is desired (sensitivity 85%, specificity 87%). Furthermore, the gene markers of the present invention perform significantly better than serum AFP, and can distinguish the BCLC stage A HCC patients from CHB patients.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method of diagnosing liver cancer in a subject, the method comprising determining in a sample obtained from the subject the gene expression level of at least one marker gene selected from the group consisting of the tumor necrosis factor, alpha-induced protein 3 (TNFAIP3) gene, the amphiregulin (AREG) gene and the GTPase IMAP family member 5 (GIMAP5) gene.

2. The method of claim 1, comprising determining the expression level of at least two of the marker genes selected from the group consisting of the TNFAIP3 gene, the AREG gene and the GIMAP5 gene.

3. The method of claim 2, comprising determining the expression level of all three of the TNFAIP3 gene, the AREG gene and the GIMAP5 gene.

4. The method of any of the preceding claims, wherein the liver cancer is hepatocellular carcinoma (HCC).

5. The method of any of the preceding claims, wherein the subject is a human.

6. The method of claim 5, wherein the human does not show signs of HCC.

7. The method of any of the preceding claims, wherein the determined expression level is compared to a control sample.

8. The method of claim 7, wherein an increased expression level in the sample of the subject relative to the control sample is indicative of a risk of developing HCC.

9. The method of any of claims 5 to 8, comprising distinguishing a subject suffering from HCC from a subject suffering from Chronic Hepatitis B

10. The method of claim 9, wherein the HCC is Barcelona Clinic Liver Cancer (BCLC) stage A HCC.

11. The method of any of claims 5 to 10, wherein the human has liver cirrhosis.

12. The method of any of the preceding claims, wherein the sample comprises a blood cell or liver tissue.

13. The method of claim 12, wherein the blood cell is a leukocyte.

14. The method of any of the preceding claims, wherein determining the gene expression level is carried out using a nucleic acid amplification assay.

15. The method of claim 14, wherein the amplification assay is a quantitative PCR assay or a real time PCR assay.

16. A method of assessing the risk of a subject having liver cirrhosis of developing liver cancer, the method comprising determining in a sample obtained from the subject the gene expression level of at least one marker gene selected from the group consisting of the tumor necrosis factor, alpha-induced protein 3 (TNFAIP3) gene, the amphiregulin (AREG) gene and the GTPase IMAP family member 5 (GIMAP5) gene.

17. The method of claim 16, wherein the liver cancer is hepatocellular carcinoma (HCC).

18. The method of claim 16 or 17, wherein the subject is a human.

19. The method of claim 18, wherein the human does not show signs of HCC.

20. The method of any of claims 16 to 19, wherein an increased expression level in the sample of the subject relative to the control sample is indicative of a risk of developing HCC.

21. The method of claim 20, which comprises monitoring the subject for the development of HCC in case of an increased expression level.

22. A method of diagnosing liver cancer in a subject, the method comprising determining in the sample obtained from the subject the presence or amount of at least one marker protein selected from the group consisting of tumor necrosis factor, alpha-induced protein 3 (TNFAIP3, SwissProt accession number: P21580), amphiregulin (AREG, SwissProt accession number P15514) and GTPase IMAP family member 5 (GIMAP5, SwissProt accession number Q96F15).

23. A kit for the diagnosis of liver cancer by determining the expression level of at least one marker gene selected from the group consisting of the tumor necrosis factor, alpha-induced protein 3 (TNFAIP3) gene, the amphiregulin (AREG) gene and the GTPase, IMAP family member 5 (GIMAP5) gene, the kit comprising one or more oligonucleotides complementary to at least one of the marker gene nucleic acid molecule.

24. The kit of claim 23, comprising two kinds of one or more oligonucleotides, wherein each kind of oligonucleotide is complementary to one of at least two of the marker gene nucleic acid molecules.

25. The kit of claim 23 or 24, comprising three kinds of one or more oligonucleotides, wherein each kind of oligonucleotide is complementary to one at of the three marker gene nucleic acid molecules.

26. The kit of any of claims 23 to 25, wherein the oligonucleotides are oligonucleotide probes.

27. The kit of claim 26, wherein the oligonucleotides probes are amplification primers.

28. The kit of claim 27, wherein said amplification primers are suitable to amplify a marker nucleic acid molecule in an amplification step.

29. The kit of any of claims 23 to 28, wherein the oligonucleotides are up to about 30, about 60, or about 100 nucleotides in length.

30. The kit of any of claims 26 to claim 29, wherein said oligonucleotide probes are labelled.

31. The kit of claim 30, wherein the label is a radioactive, fluorescent, chemoluminescent, affinity, or enzymatic label.


SEQUENCE LISTING (21 sequences)

SEQ ID NO: 1 - DNA, 21 bp, Artificial Sequence
  forward primer for NFKBIZ
tcctgttgcacatccgaagtc

SEQ ID NO: 2 - DNA, 21 bp, Artificial Sequence
  reverse primer for NFKBIZ
tccatcagacaacgaatcggg

SEQ ID NO: 3 - DNA, 20 bp, Artificial Sequence
  forward primer for GIMAP8
gggtcgctctccggccattc

SEQ ID NO: 4 - DNA, 21 bp, Artificial Sequence
  reverse primer for GIMAP8
caggctcccgcttgttctggg

SEQ ID NO: 5 - DNA, 22 bp, Artificial Sequence
  forward primer for GIMAP5
gtgcagctgagtcatggagctt

SEQ ID NO: 6 - DNA, 25 bp, Artificial Sequence
  reverse primer for GIMAP5
ttctctccagaaacggttgttgtgc

SEQ ID NO: 7 - DNA, 24 bp, Artificial Sequence
  forward primer for AREG
gtggtgctgtcgctcttgatactc

SEQ ID NO: 8 - DNA, 23 bp, Artificial Sequence
  reverse primer for AREG
tcaaatccatcagcactgtggtc

SEQ ID NO: 9 - DNA, 20 bp, Artificial Sequence
  forward primer for CD83
tgcactctgcaggacccgga

SEQ ID NO: 10 - DNA, 25 bp, Artificial Sequence
  reverse primer for CD83
tgtagccgtgcaaacttacaagtga

SEQ ID NO: 11 - DNA, 23 bp, Artificial Sequence
  forward primer for NFKBIA
ctccgagactttcgaggaaatac

SEQ ID NO: 12 - DNA, 23 bp, Artificial Sequence
  reverse primer for NFKBIA
gccattgtagttggtagccttca

SEQ ID NO: 13 - DNA, 23 bp, Artificial Sequence
  forward primer for GIMAP6
gtcttcgagtctaaactcagcac

SEQ ID NO: 14 - DNA, 21 bp, Artificial Sequence
  reverse primer for GIMAP6
tgggtgtgtcaatcacctcaa

SEQ ID NO: 15 - DNA, 21 bp, Artificial Sequence
  forward primer for TNFAIP3
ttgtcctcagtttcgggagat

SEQ ID NO: 16 - DNA, 22 bp, Artificial Sequence
  reverse primer for TNFAIP3
ttctcgacaccagttgagtttc

SEQ ID NO: 17 - DNA, 20 bp, Artificial Sequence
  forward primer for GIMAP4
gcccaatacggcagtatgag

SEQ ID NO: 18 - DNA, 20 bp, Artificial Sequence
  reverse primer for GIMAP4
cctgctccggttttacccac

SEQ ID NO: 19 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 20 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 21 - protein, 408 aa, Homo sapiens
MNNMLDIWQSRLQEHIKETRTYMKYMLNDHLVIVLIFFLAGAASWYSKWIRDIPAHFPSFWVMAVLFSLVLTSSYVRTLLKEADLVFLLPLEAKMEPYLKQAFVYSYVSQLFPLIALSIVAMPLYFAVTPGASLVSYAAVFVQLLLLKAWNQVMEWRTTFQNDRSMKRMDVIIRFAANTLVLYFVFQSVYMYALLVYVIMAVLYLYMSSAAKRKTFKWESHIESELRRKQRFYRIANLFTDVPHLRKQAKRRAYLDFLLRLVPFEQRKTFAYMFTRAFLRSSDYLGILVRLTIVFALIIMYVSASPLIAAVLTVFAIFITGIQLLPLFGHFDHLALQELYPVQKETKLKSYFSLLKTALSIQALLMSVASAYAAGLTGFLYALIGSAVLIFVVLPAYMTTRLKKHGKL